(19) United States
(12) Patent Application Publication
(10) Pub. No.: US 2021/0236425 A1
(43) Pub. Date: Aug. 5, 2021

(54) NEW TREATMENT OF INTERSTITIAL LUNG DISEASES

(71) Applicant: INIM PHARMA AB, Stockholm (SE)

(72) Inventors: Carl-Johan DALSGAARD, Stockholm (SE); Adam FEILER, Hässelby (SE); Chunfang ZHOU, Stockholm (SE); Paulina PASZKIEWICZ, Stockholm (SE); Xin XIA, Bandhagen (SE)

(21) Appl. No.: 17/052,649

(22) PCT Filed: May 3, 2019
(86) PCT No.: PCT/GB2019/051237

§ 371 Date: Nov. 3, 2020

(30) Foreign Application Priority Data
May 3, 2018 (GB) 1807286.8

(51) Int. Cl. A61K 9/14; A61K 9/00; A61K 31/45; A61K 9/16

(52) U.S. Cl. CPC A61K 9/143; A61K 9/1682; A61K 31/45; A61K 9/0073

(57) ABSTRACT

According to the invention, there is provided a pharmaceutical composition suitable for administration to the lung, which composition comprises a plurality of amorphous nanoporous silica particles, in which one or more immunomodulatory imide drug is loaded into the pores of said particles, and wherein the silica particles have: (a) a mass median aerodynamic diameter that is between about 0.1 μm and about 10 μm; and (b) a geometric standard deviation that is less than about 4, for use in the treatment of an interstitial lung disease by pulmonary administration. Preferred immunomodulatory imide drugs include thalidomide. Interstitial lung diseases that may be mentioned include idiopathic pulmonary fibrosis and sarcoidosis.

### FIELD OF THE INVENTION

This invention relates to a new pharmaceutical use.

### BACKGROUND AND PRIOR ART

Interstitial lung diseases (ILDs) are a group of lung diseases that affect the interstitium, characterised by tissue around alveoli becoming scarred and/or thickened, and so inhibiting the respiratory process.

ILDs are distinct from obstructive airway diseases (e.g. chronic obstructive airway disease (COPD) and asthma), which are typically characterized by narrowing (obstruction) of bronchi and/or bronchioles. ILDs may be caused by injury to the lungs, which triggers an abnormal healing response but, in some cases, these diseases have no known cause. ILDs can be triggered by chemicals (silicosis, asbestosis, certain drugs), infection (e.g. pneumonia) or other diseases (e.g. rheumatoid arthritis, systemic sclerosis, myositis or systemic lupus erythematosus).

The most common ILDs are idiopathic pulmonary fibrosis (IPF) and sarcoidosis, both of which are characterised by chronic inflammation and reduced lung function.

Sarcoidosis is a disease of unknown cause that is characterised by collections of inflammatory cells that form lumps (granulomas), often beginning in the lungs (as well as the skin and/or lymph nodes, although any organ can be affected). When sarcoidosis affects the lungs, symptoms include coughing, wheezing, shortness of breath, and/or chest pain.

Treatments for sarcoidosis are patient-specific. In most cases, symptomatic treatment with non-steroidal anti-inflammatory drugs (NSAIDs) is possible, but for those presenting lung symptoms, glucocorticoids (e.g. prednisone or prednisolone), antimetabolites and/or monoclonal anti-tumor necrosis factor antibodies are often employed.

IPF on the other hand is a chronic lung disease characterized by a progressive and irreversible decline in lung function caused by scarring of the lungs. Symptoms typically include cough and shortness of breath. Although less prevalent than asthma and COPD, mortality rates from IPF are much higher (e.g. 5 times higher than that of asthma, despite asthma being 100 times more prevalent).

Current treatment of IPF includes oxygen supplementation. Medications that have been tried include pirfenidone or nintedanib, but only with limited success in slowing the progression of the disease. Further, both of these drugs commonly cause (predominantly gastrointestinal) side-effects.

IPF affects about 5 million people globally. Average life expectancy after diagnosis is around four years.

There are drawbacks associated with all of the aforementioned ILD drug treatments and there is a real clinical need for safer and/or more effective treatments.

Immunomodulatory imide drugs (IMIDs) are a class of immunomodulatory drugs that contain an imide group. The drug class includes thalidomide and analogues thereof, such as lenalidomide and pomalidomide.

Primary medical uses of IMIDs include the treatment of cancers, such as multiple myeloma and myelodysplastic syndrome (a precursor condition to acute myeloid leukaemia), as well as certain autoimmune diseases (including erythema nodosum, a complication of leprosy). Off-label uses include other forms of cancer, such as Hodgkin's lymphoma and prostate cancer, as well as other conditions like primary myelofibrosis. Cyclodextrin-based thalidomide formulations for the treatment of nosebleeds in hereditary hemorrhagic telangiectasia are also known (see Colombo et al, Int. J. Pharm., 514, 229 (2016)).

Thalidomide's infamous history of causing birth defects following its use as an antiemetic during pregnancy is well known.

In addition to the above, there are published reports of thalidomide's potential use as a systemic treatment of ILDs, such as idiopathic PF (see Zhao et al, Clin. Exp. Immunol., 157, 310 (2000), Zhang and Yang, Journal of Xi'an Jiatong University (Medical Sciences), 33, 622 (2012), Horton et al, Ann. Intern. Med., 157, 398 (2012) and Haraf et al, Am. J. Ther., 1 (2017)), bleomycin-induced PF in animals (Tabata et al, J. Immunol., 179, 708 (2007) and Don et al, Am. J. Transl. Res., 9, 4390 (2017)), and pulmonary sarcoidosis (Carlesimo et al, J. Am. Acad. Dermatol., 32, 866 (1995) and Fazzi et al, Biomedicin & Pharmacotherapy, 66, 300 (2012)). Lenalinomide has also been suggested for use systemically in pulmonary sarcoidosis (Jafari et al, Chest, 148, e35 (2015)).

There is no clear and direct disclosure or suggestion of the possibility of administering IMIDs by the pulmonary route for any indication, let alone in the specific, topical treatment of ILDs.

Particles comprising nanoporous (mesoporous) silica materials have been disclosed for use in general pharmaceutical and cosmetic applications in inter alia international patent application WO 2012/035074. Here, poorly soluble active ingredients are incorporated within nanopore channels of the silica particles. Similarly, formulations comprising lenalidomide in various porous carriers including silica have been described in WO 2016/097030. In Ao et al (Braz. J. Med. Res., 51, 1 (2018)), low density lipopeptide modified silica nanoparticles loaded with docetaxel and thalidomide for use in chemotherapy of liver cancer are disclosed. There is no suggestion in any of these documents of the use of such compositions in delivery of active ingredients to the lungs.

The use of porous materials for potential delivery of active ingredients to the lung has been disclosed. For example, U.S. Pat. Nos. 6,254,854, 6,740,310 and 7,435,408 all disclose polymeric materials, such as polyanhydrides and copolymers poly(lactic acid) grafted to amino acids; and international patent application WO 03/043586 discloses polymeric nanoparticles. Tartula et al (J. Drug Target., 19, 900 (2011)), Li et al (Nanomedicine, 11, 1377 (2015) and Wang et al, Nanoscale Research Letters, 12, 66 (2017)) disclose mesoporous silica nanoparticles for inhaled delivery of drugs. Finally, international patent application WO 03/011251 discloses mesoporous silicon carriers for pulmonary delivery.

Inhalation devices that are typically employed to administer active compounds to the lung include metered dose inhalers (MDIs), dry powder inhalers (DPIs) and soft mist inhalers (SMIs). DPIs may be divided into low, medium and high resistant DPIs.

The efficiency of DPIs, for example, is affected by two main forces 1) an inspiration air flow (IAF), which depends on a flow generated by the patient, and 2) a turbulence produced by the device.

A balance between these two forces is important for optimal performance of a device. If the IAF is too high, most of the drug is lost in the upper lung, i.e. the throat and the trachea. On the other hand, with most DPI-administered formulations, if the IAF is too low, more drug may be delivered in the lower lung (the bronchi and alveoli), but in a manner where there is often poor disaggregation of particles, and therefore dispersion of the drug in the lung.

Typical fixed-dose drug combinations for pulmonary delivery require powder homogeneity to deliver a uniform dose of drug to patients. This is often attempted by a simple blend of micronized drugs with coarse carrier particles.

In a MDI, the pharmaceutical composition is typically present in a liquid form, as a solution or suspension in a propellant, such as a hydrocarbon, a fluorocarbon or a hydrogen-containing fluorocarbon. In such systems it is often difficult to prevent dissolution of a bioactive compound from the particle or to prevent leakage of the compound from the drug-containing particle.

Typically, solvents or surfactants are employed with a view to imparting stability to the suspension of drug particles. The compound needs to have a low solubility in the solvents that are used.

In attempting to formulate specific mesoporous silica materials with specific properties with a view to inclusion of IMIDs, such as thalidomide, for pulmonary delivery, an undesirable agglomeration resulted, which could not be alleviated with conventional lubricant excipients. Surprisingly, incorporation of IMIDs, such as thalidomide, into the resultant silica particles resulted in deagglomeration of the resultant loaded particles, which was not expected. This renders the resultant loaded silica particles of potential utility in the pulmonary delivery of such active ingredients.

We have also found that IMIDs show an excellent solubility profile when presented in this way, which renders such compositions of potential utility in the topical treatment of ILDs, such as IPF, by pulmonary administration.

### DISCLOSURE OF THE INVENTION

According to the invention, there is provided a pharmaceutical composition suitable for administration to the lung, which composition comprises a plurality of amorphous nanoporous (mesoporous) silica particles, in which one or more IMID is loaded into the pores of said particles, and wherein the silica particles have:
(a) a mass median aerodynamic diameter (MMAD) that is between about 0.1 μm and about 15 μm (e.g. about 10 μm); and
(b) a geometric standard deviation (GSD) that is less than about 4,
   for use in the pulmonary treatment of an ILD.

Such compositions for use in the treatment of ILD as defined above are hereinafter referred hereinafter together as “compositions of the invention”.

The loaded silica particles of the compositions of the invention may also have a mass density that is less than about 0.4 g/cm³, for example between about 0.15 and about 0.35 g/cm³.

Mass median aerodynamic diameter (MMAD) will be understood by those skilled in the art to mean the diameter at which 50% of the particles by mass are larger and 50% are smaller (see US Pharmacopeia at <601>). MMAD may be readily determined by those skilled in the art, for example by plotting on log probability paper the percentages of mass that is less than the stated aerodynamic diameters versus the aerodynamic diameters. The MMAD is taken as the intersection of the line with the 50% cumulative percent.

MMAD of the particles may be varied depending on the preferred and/or intended site of delivery of the IMID compound. A MMAD that may be mentioned is between about 5 μm and about 15 μm. However, it is preferred that the MMAD of particles in compositions of the invention is between about 0.5 μm and about 8 μm, such as between about 1 μm and about 7 μm, for example between about 2 μm and about 6 μm, more preferably between about 3 μm and about 5 μm. This will mean that particles will tend to deposit primarily in the bronchioli.

GSD will be understood by those skilled in the art to be a measure of the spread of an aerodynamic particle size distribution. It is typically calculated as follows as:

(d₉₀/d₁₀)^{1/2}

wherein d₉₀ and d₁₀ represent the diameters at which 90% and 10%, respectively, of the aerosol mass are contained, in diameters less than these diameters.

It is preferred that the GSD of particles in compositions of the invention is less than about 2.5, such as less than about 2.2, e.g. less than about 2.0, including less than about 1.8, or more preferably less than about 1.5, such as between about 1 and about 1.5.

Other parameters that may be used to define particles include mass density and the fine particle fraction (FPF). The FPF is the proportion of particles that have a diameter below about 5 μm. Preferred FPFs are at least about 75%, such as at least about 80%, including at least about 85%, e.g. at least about 90%, such as at least about 95%, at least about 98%, and up to at least about 99%, at least about 99.9% or about 100%.

The silica particles may be separated and classified into the desired particle size range by any process known to those skilled in the art. For example, particles may be separated using cyclonic separation, by way of an air classifier, by elutriation, sedimentation and/or by sieving using one or more sieves or filters to obtain particles within a desired size range. Preferably, particles within a desired size range are obtained using an air classifier.

The silica particles that are employed in compositions of the invention may have a pore size that is between about 1 nm (e.g. about 2 nm) and about 100 nm (e.g. about 50 nm). Porous silica particles of compositions of the invention may have a preferred average pore size that is between about 3 nm (e.g. about 5 nm, such as about 8 nm) and about 30 nm (e.g. about 20 nm, such as about 15 nm), such as between about 11 nm and about 14 nm, such as about 13 nm and more preferably about 12 nm. Such particles may also possess a pore volume that is between about 0.1 cm³/g (e.g. about 0.2 cm³/g) and about 3 cm³/g and/or may possess a surface area that is between about 40 m²/g (e.g. about 150 m²/g) and about 1200 m²/g. All of these parameters may be determined by routine techniques, such as nitrogen adsorption isotherm (Brunauer, Emmett and Teller (BET)), and mercury inclusion, methods.

Shapes of the porous particles may be controlled by the process of manufacture. Shape may be important for the incorporation and dissolution of the IMID. Thus, although silica particles may potentially be any shape (e.g. gyroids, rods, fibers, pseudo-spheres, cylinders, core-shells) in compositions of the invention, they are preferably essentially spherical. By “essentially spherical”, we mean that they may possess an aspect ratio smaller than about 20, more preferably less than about 10, such as less than about 4, and especially less than about 2, and/or may possess a variation in radii (measured from the centre of gravity to the particle surface), in at least about 90% of the particles that is no more than about 20% of the average value, such as no more than about 10% of that value, for example no more than about 5% of that value.

Porous silica particles may be loaded with one or more IMIDs by any suitable process known to those skilled in the art. For example, particles may be loaded by way of a solvent evaporation technique, for example as described hereinafter, impregnation, for example using a melt, use of supercritical CO₂, shear mixing, co-grinding, spray-drying or freeze-drying. Well known equipment, such as a fluidized bed may be used. A preferred technique is solvent evaporation.

Loading the IMID into the silica particles means that the IMID is loaded into the nanopores of the particles. It is preferred that the pores of the silica particles are loaded such that between about 0.1 and about 60%, preferably up to about 50%, such as up to about 45% of the total weight of the loaded particles is IMID and, optionally, other pharmaceutical excipients, diluents or additives. In the alternative, it is preferred that up to about 80%, such as up to about 90%, e.g. up to about 100% of the pores of the silica particles are loaded with IMID and, optionally, other pharmaceutical excipients, diluents or additives. The entire mass of IMID does not have to be loaded into the pores of the particles and may otherwise be attached to the surfaces of the particle.

It is further preferred that the IMID is presented within the pores of the particles of compositions of the invention in an essentially amorphous state. By “essentially amorphous”, we mean that the IMID is no more than about 5%, such as no more than about 2%, for example no more than about 1%, e.g. no more than about 0.5%, and preferably no more than about 0.1% crystalline.

Presenting IMID in an amorphous state within the pores of the particles of compositions of the invention means that the latter are capable of delivering a consistent and/or uniform dose of IMID, which is independent of solubility, after administration to the lung. We have found that, by incorporating IMID into the pores of the particles of the compositions of the invention, it is possible to ensure that IMID remains in the amorphous state during and after manufacture, under normal storage conditions, and during use.

By this, we include that the IMID compound can be stored in the form of a composition of the invention, optionally in admixture with pharmaceutically acceptable carriers, diluents or adjuvants, under normal storage conditions, with an insignificant degree of solid state transformation (e.g. crystallisation, recrystallisation, loss of crystallinity, solid state phase transition, hydration, dehydration, solvatisation or desolvatisation).

Examples of “normal storage conditions” include temperatures of between minus 80 and plus 50° C. (preferably between 0 and 40° C. and more preferably ambient temperature, such as between 15 and 30° C.), pressures of between 0.1 and 2 bars (preferably atmospheric pressure), relative humidities of between 5 and 95% (preferably 10 to 60%), and/or exposure to 460 lux of UV/visible light, for prolonged periods (i.e. greater than or equal to six months). Under such conditions, IMID may be found to be less than about 15%, more preferably less than about 10%, and especially less than about 5%, solid-state transformed. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

We prefer that the amorphous porous silica particles are biodegradable mesoporous silica.

The term “biodegradable” means that the silica particles are dissolvable. Accordingly, a preferred embodiment of the invention is that the silica is a synthetic amorphous silica.

In order to be completely dissolvable, the silica particles of the compositions of the invention must be amorphous and therefore entirely non-crystalline (and remain so under normal storage conditions as hereinbefore defined), by which we mean that no crystallinity is detectable by standard techniques, such as XPRD. This is especially important considering the indications in which the compositions of the invention are intended to be used, in which injury by crystalline silica or other agents may be one of the causes of ILDs, such as PF.

Amorphous silica is less sensitive to humidity when compared to dry crystalline powder compositions that are typically used in pulmonary delivery of active ingredients.

Amorphous silica particles may be manufactured by any process known in the art. In one embodiment, porous particles may be manufactured by cooperative self-assembly of silica species and organic templates such as cationic surfactants such as alkyltrimethylammonium templates with varying carbon chain lengths, and counterions such as cetyltrimethylammonium chloride (CTA+Cl− or CTAC) or cetyltrimethylammonium bromide (CTA+Br− or CTAB), or non-ionic species such as diblock and triblock polymer species, such as copolymers of polyethylene oxide and polypropylene oxide for example Pluronic 123 surfactant.

The formation of mesoporous silica particles occurs following the hydrolysis and condensation of silica precursors which can include alkylsilicates such as tetraethylorthosilcate (TEOS) or tetramethylorthosilicate (TMOS) in solution or sodium silicate solution. The mesoporous silica particle size can be controlled by adding suitable additive agents, e.g. inorganic bases, alcohols including methanol, ethanol, propanol, and other organic solvents, such as acetone, which affect the hydrolysis and condensation of silica species.

The pore size can be influenced by hydrothermal treatment of the reaction mixture such as heating up to 100° C. or even above and also with the addition of swelling agents in the form of organic oils and liquids that expand the surfactant micelle template.

After condensation of the silica matrix, the templating surfactant can be removed by calcination typically at temperatures from 500° C. up to 650° C. for several hours which burns away the organic template resulting in a porous matrix of silica. The template may alternatively be removed by extraction and washing with suitable solvents such as organic solvents or acidic of basic solutions.

In another embodiment, the porous silica particles may be manufactured by a sol-gel method comprising a condensation reaction of a silica precursor solution, such as sodium silicate or an aqueous suspension of silica nanoparticles as an emulsion, with a non-miscible organic solution, oil, or liquid polymer in which droplets are formed by for example stirring or spraying the solution, followed by gelation of the silica by means of change in pH and/or evaporation of the aqueous phase.

The porosity of the particles here are formed either by exclusion due to the presence of the non-miscible secondary phase or by the jamming of the silica nanoparticles during evaporation.

Such particles may further be treated by heating to induce condensation of the silica matrix and washing to remove the non-miscible secondary phase. Furthermore, the particles may be treated by calcination to strengthen the silica matrix.

In another embodiment, the porous particles may be manufactured as porous glass through a process of phase separation in borosilicate glasses (such as SiO₂—B₂O₃—Na₂O), followed by liquid extraction of one of the formed phases through the sol-gel process, or simply by sintering glass powder. During a thermal treatment, typically between 500° C. and 760° C., an interpenetration structure is generated, which results from a spinodal decomposition of the sodium-rich borate phase and the silica phase.

The porous particles may also be manufactured using a fumed process. In this method, fumed silica is produced by burning silicon tetrachloride in an oxygen-hydrogen flame producing microscopic droplets of molten silica which fuse into amorphous silica particles in three-dimensional secondary particles which then agglomerate into tertiary particles. The resulting powder has an extremely low bulk density and high surface area.

According to a further aspect of the invention there is a provided a process for the production of a composition of the invention, which process comprises:
(a) separating silica particles to obtain particles having an MMAD and/or a GSD within the ranges described herein; followed by
(b) loading the obtained particles with an IMID as described herein.

The process described herein for production of compositions of the invention has the advantage that it allows the production of particles with sizes that enable better control of the site of deposition of the particles in the lung, so enabling accurate tailoring of site-specific lung delivery (e.g. improved delivery to the deep lung) compared to prior art inhalation formulations comprising other drugs. The process described herein also reduces manufacturing costs compared to processes in which separation is conducted after loading particles with a bioactive compound. This may improve the yield and efficiency of the manufacturing process. The process also provides for a higher drug loading of the bioactive compounds in final compositions of the invention.

In view of the particle size of the silica particles of the compositions of the invention, particle aggregation was expected. Aggregation of dry particles in the micron-sized range is a well-known phenomenon in particle and powder processing. Aggregation is caused by numerous attractive (ubiquitous) forces, such as van der Waals forces and/or electrostatic interactions. In many cases, particle aggregation causes unwanted problems such as poor handling and flowability and sticking to containers.

Particles within the size range mentioned herein are also often prone to aggregation in air due to the large surface area to volume ratio.

Particle aggregation is a serious hurdle for pulmonary delivery, given that the particle size is critical to ensure correct distribution of the particles in the lung. Aggregation of particles would be expected to lead to accumulation in the throat and upper airways thereby limiting the effectiveness of the formulation. Additionally, aggregation of particles or sticking of particles in the capsules during inhalation is a severe limitation.

As described hereinafter, when small amounts of pre-loaded silica particles that might be considered to be suitable for inhalation were investigated, as expected, the particles aggregated and had poor flow properties. When attempts were made to use common excipients that are normally employed to reduce particle aggregation, the flow properties of the particles did not improve.

However, as also described hereinafter, when the same particles were loaded with an IMID (e.g. thalidomide), the flow properties of the particles was dramatically improved without the addition of further excipients.

Compositions of the invention thus have unexpectedly good flow properties which renders them suitable for pulmonary delivery.

The term “ILD” will be understood by those skilled in the art to include any pulmonary condition characterized by an abnormal healing response, including chronic inflammation, reduced lung function and/or scarring, irrespective of the cause, such as sarcoidosis, and PF, especially IPF.

According to a further aspect of the invention there is provided a method of treatment of an ILD, which method comprises the pulmonary administration of a pharmacologically-effective amount of an IMID in the form of a composition of the invention to a patient in need of such treatment.

“Patients” include mammalian (particularly human) patients. Human patients include both adult patients as well as paedeatric patients, the latter including patients up to about 24 months of age, patients between about 2 to about 12 years of age, and patients between about 12 to about 16 years of age. Patients older than about 16 years of age may be considered adults for purposes of the present invention. These different patient populations may be given different doses of IMID.

IMIDs include lenalidomide, pomalidomide and, especially, thalidomide. IMIDs may be administered in the form of racemates, single enantiomers and/or pharmaceutically-acceptable salts.

Pharmaceutically-acceptable salts of IMIDs include base addition salts and preferably acid addition salts. Such salts may be formed by conventional means, for example by reaction of a free acid or, preferably, free base form of an active ingredient with one or more equivalents of an appropriate acid or base as appropriate, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of an active ingredient in the form of a salt with another counter-ion, for example using a suitable ion exchange resin.

Pulmonary delivery means compositions of the invention are adapted for delivery to the lungs by direct inhalation, and thereby giving rise to the direct topical treatment by IMIDs of ILDs in the lungs.

Administration of IMID is preferably intermittent. The mode of administration may also be determined by the timing and frequency of administration, but is also dependent, in the case of the treatment of ILDs, on the severity of the condition.

Compositions of the invention may also impart, or may be modified to impart, an immediate, or a modified, release of IMIDs.

Compositions of the invention may be combined with other excipients that are well known to those skilled in the art for pulmonary delivery of active ingredients. For example, optional excipients may include propellants; surfactants, such as poloxamers; glidants/lubricants, such as magnesium stearate or silica nanoparticles; sugars or sugar alcohols, such as lactose, glucose, mannitol or trehalose; lipids, such as DPPC, DSPC, DMPC, cholesterol; amino acids, such as leucine or trileucine; cyclodextrins, hydroxypropylated chitosan, poly-lactic-co-glycolic acid (PLGA); antioxidants; humidity regulators and the like, though such are by no means essential. Indeed, we have found that, in the pulmonary delivery of compositions of the invention, fewer additional excipients are needed, which may reduce cost of manufacture.

Inhalation devices that may be employed to administer compositions of the invention to the lung include MDIs, SMIs and DPIs, including low, medium and high resistant DPIs.

Compositions of the invention may form stable compound suspensions when suspended in solvents that are typically employed in MDIs. The loaded silica particles may be well-dispersed in different solvents and may be further modified to prevent dissolution or leakage of drug into the solvent before delivery to the target site or lung.

In view of the fact that, as mentioned hereinbefore, compositions of the invention have unexpectedly good flow properties, this minimizes the need for disaggregation of the particles by increased IAF and turbulence produced by the inhalation device. This in turn improves that the balance between the two forces discussed hereinbefore, and thus improves delivery of IMID to the lower lung without loss of drug in the upper lung. This further reduces the dependence on the inhalation device that is employed.

According to a further aspect of the invention, there is further provided a drug delivery device adapted for delivery of active ingredients to the lung, which delivery device comprises a composition of the invention.

The delivery device may be a MDI, a DPI or a SMI. When used in, in particular, a MDI, the composition of the invention is optionally mixed with a propellant, which propellant has a sufficient vapour pressure to form aerosols upon activation of the delivery device. The propellant may be selected from the group a hydrocarbon, a fluorocarbon, a hydrogen-containing fluorocarbon and a mixture thereof.

The above-mentioned excipients may be commercially-available or otherwise are described in the literature, for example, Remington The Science and Practice of Pharmacy, 19th ed., Mack Printing Company, Easton, Pa. (1995) and the documents referred to therein, the relevant disclosures in all of which documents are hereby incorporated by reference. Otherwise, the preparation of suitable pulmonary formulations may be achieved non-inventively by the skilled person using routine techniques.

Similarly, the amount of IMID in the formulation will depend on the severity of the condition, and on the patient, to be treated, but may be determined by the skilled person.

For example, suitable lower daily doses (calculated as the free base) of thalidomide in adult patients (average weight e.g. 70 kg), may be about 0.01 mg, such as about 0.1 mg, for example about 1 mg, or about 5 mg, per day. Suitable upper limits of daily dose ranges of e.g. thalidomide may be about 200 mg, such as about 50 mg, including about 25 mg, such as about 10 mg.

All of the above doses are calculated as the free base and, again, doses may be split into multiple individual doses per day. Inhaled doses may be given between once and four times daily, preferably three times daily and more preferably twice daily. Alternatively, inhaled doses may be given between once and four times weekly, for example every other day.

In any event, the medical practitioner, or other skilled person, will be able to determine routinely the actual dosage, which will be most suitable for an individual patient, depending on the severity of the condition and route of administration. The above-mentioned dosages are exemplary of the average case; there can, of course, be individual instances where higher or lower dosage ranges are merited, and such are within the scope of this invention.

The dose administered to a mammal, particularly a human, in the context of the present invention should be sufficient to effect an appropriate response in the mammal (e.g. human) over a reasonable timeframe (as described hereinbefore). One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease, as well as genetic differences between patients.

Although intended for inhalation, compositions of the invention may be co-administered with other pharmaceutical formulations comprising different (or the same) active ingredients that are intended for the treatment of ILDs (whether administered pulmonarily, orally, or otherwise). For example, uses and methods that involve pulmonary administration of compositions of the invention may be combined with one or more treatments comprising other active ingredients that are useful in the treatment of ILDs (or a peroral treatment comprising one or more IMID).

Relevant active ingredients for use in IPF include, for example, anti-fibrotics, such as nintedanib and pirfenidone; corticosteroids, such as cortisone and prednisone; inflammation suppressants, such as cyclophosphamide; other immunosuppressants, such as azathioprine and mycophenolate mofetil; and antioxidants, such as N-acetylcysteine. Relevant active ingredients for use in sarcoidosis include, for example, corticosteroids, such as cortisone, prednisone and prednisolone; antimetabolites; immune system suppressants, such as methotrexate, azathioprine, leflunomide, mycophenoic acid/mycophenolate mofetil, cyclophosphamide; aminoquinolines; monoclonal anti-tumor necrosis factor antibodies, such as infliximab and adalimumab; and painkillers, such as ibuprofen and paracetamol; cough suppressants and/or expectorants.

Relevant patients may thus also (and/or may be already) be receiving such therapy for the treatment of their ILD based upon administration of one or more of such active ingredients, by which we mean receiving a prescribed dose of one or more of those active ingredients mentioned herein, prior to, in addition to, and/or following, treatment with IMID.

Pharmaceutically-acceptable salts, and doses, of other active ingredients useful in the treatment of ILDs include those that are known in the art and described for the drugs in question to in the medical literature, such as Martindale—The Complete Drug Reference (35^{th} Edition) and the documents referred to therein, the relevant disclosures in all of which documents are hereby incorporated by reference.

Wherever the word “about” is employed herein, for example in the context of amounts, i.e. absolute amounts such as sizes (aerodynamic diameters, particle sizes and pore sizes), doses, weights or concentrations of (e.g. active) ingredients, densities or time periods; or relative amounts including percentages, standard deviations and aspect ratios, it will be appreciated that such variables are approximate and as such may vary by ±10%, for example ±5% and preferably ±2% (e.g. ±1%) from the actual numbers specified. In this respect, the term “about 10%” means e.g. ±10% about the number 10, i.e. between 9% and 11%.

Compositions of the invention provide for an improved drug loading for the reasons described hereinbefore. This enables high quantities/doses of bioactive compound to be presented in compositions of the invention, and also efficient delivery of such higher doses to the desired site in the lung in a consistent/uniform manner. This in turn means that the frequency of dosing may be reduced and thus the effectiveness and efficiency of treatment as well as costs for healthcare reduced.

Furthermore, improved efficiency of deposition of the IMID compound in the lung allows for more precise lung delivery, and thus an improved therapeutic effect.

Homogeneity in terms of both carrier particle size and drug distribution within the composition may also be improved by compositions of the invention.

In addition, if desired, compositions of the invention may include additional bioactive compounds (IMID or otherwise as described hereinbefore), which may also be loaded into silica particles without substantial loss of material. This may be useful in e.g. co-therapy as described hereinbefore, and moreover may further reduce cost of manufacture.

Compositions of the invention also have the advantage that the dissolution kinetics of the IMID compound is largely independent of particle size, morphology of the compound and site of delivery in the lung. Adjusting pore size may thus be employed to tailor drug dissolution kinetics, but will be independent of the position of the particles in the lung.

The uses/methods described herein may otherwise have the advantage that, in the treatment of ILDs, they may be more convenient for the physician and/or patient than, be more efficacious than, be less toxic than, have a broader range of activity than, be more potent than, produce fewer side effects than, or that it may have other useful pharmacological properties over, similar methods (treatments) known in the prior art.

The invention is illustrated, but in no way limited, by the following example, in which

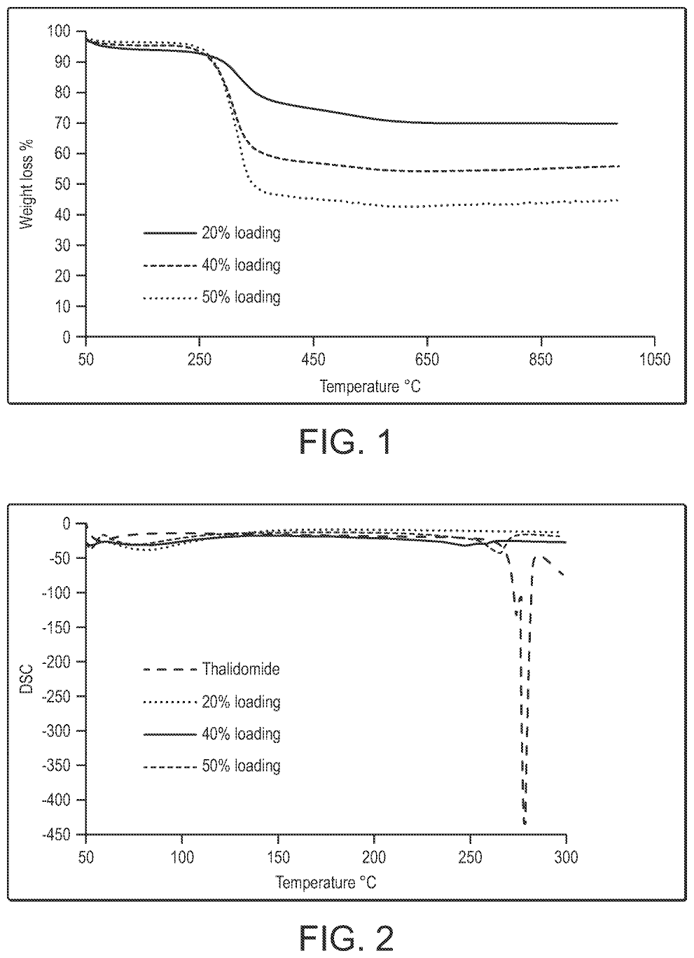

FIG. 1 is a thermogravimetric analysis (TGA) of silica particles loaded with thalidomide;

FIG. 2 is a differential scanning calorimetry (DSC) analysis characterizing the physical state of thalidomide after being loaded into silica particles;

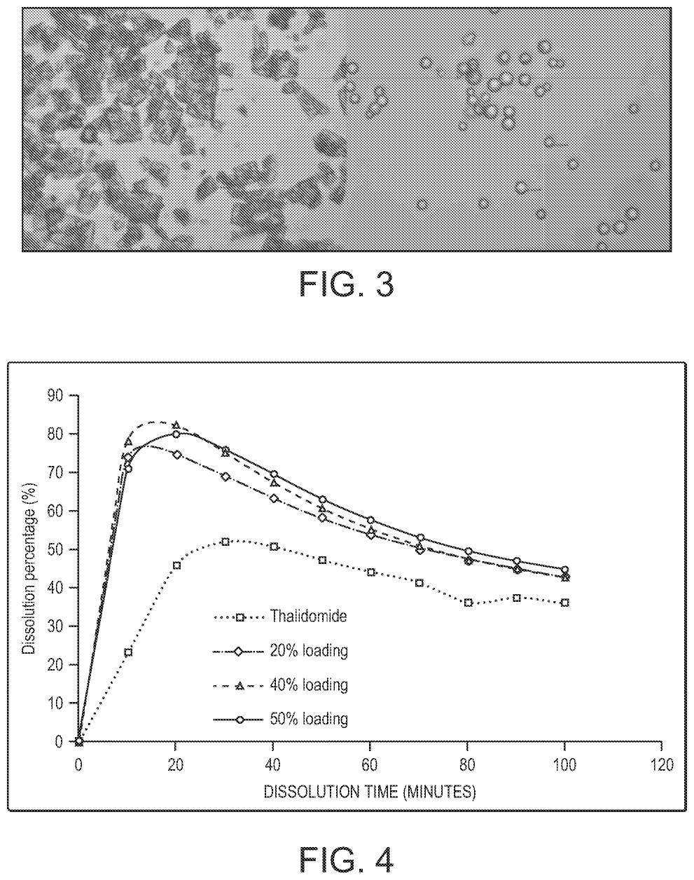

FIG. 3 is a light microscope image showing free thalidomide (on the left) and thalidomide loaded into silica particles (on the right);

FIGS. 4, 5 and 6 show in vitro release profiles for thalidomide released from silica particles in simulated lung fluid (SLF), phosphate buffered saline (PBS) and SLF with added surfactant, respectively; and

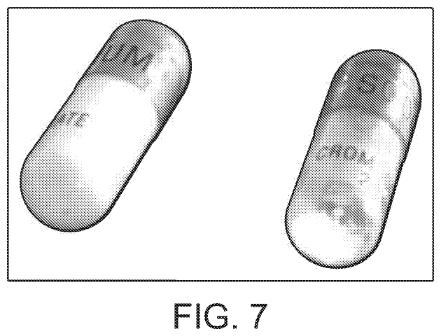

FIG. 7 shows capsules incorporating silica particles (unloaded (on the left) and loaded with thalidomide (on the right)).

### EXAMPLES

### Example 1

### Particle Manufacture I

Pluronic 123 (triblock co-polymer, EO20PO70EO20, Sigma-Aldrich; 4 g; templating agent) and 1,3,5-trimethylbenzene (TMB; mesitylene, Sigma-Aldrich; 3.3 g; swelling agent) were dissolved in 127 mL of distilled H₂O and 20 mL of hydrochloric acid (HCl, 37%, Sigma-Aldrich) while stirring at room temperature for 3 days.

The solution was preheated to 40° C. before adding 9.14 mL of tetraethyl orthosilicate (TEOS; Sigma-Aldrich). The mixture was stirred for another 10 minutes at a speed of 500 rpm, then kept at 40° C. for 24 hours, and then hydrothermally treated in the oven at 100° C. for another 24 hours. Finally, the mixture was filtered, washed and dried at room temperature.

The product was calcined to remove the surfactant template and swelling agent. The calcination was conducted by heating to 600° C. with a heating rate of 1.5° C./min and kept at 600° C. for 6 hours, followed by cooling to ambient conditions. The resultant product was a white powder comprising porous silica particles.

### Example 2

### Particle Manufacture II

A dispersion (14 wt %) of silica nanoparticles (10 nm) in water (pH 9) (400 mL) was poured into benzyl alcohol (800 mL) warmed to 50° C. and stirred at 300 rpm with an overhead stirrer (Silverson, UK) for 20 minutes.

A drop of acetic acid was added and vacuum (200 bar) was applied during heating at 80° C. to remove the aqueous phase. The resulting particles were collected by filtration and washing with acetone.

The product was calcined by heating to 600° C. with a heating rate of 1.5° C./min and kept at 600° C. for 6 hours, followed by cooling to ambient conditions. The resultant product was a white powder comprising porous silica particles in the size range 2 to 4 microns measured by scanning electron microscope (JEOL, Japan) and by electrical sensing zone method (Elzone, Micromeretics USA). The particles were further treated by refluxing in ammonium hydroxide overnight followed by filtering and refluxing in nitric acid overnight and finally filtered and washed in water and oven dried at 80° C.

### Example 3

### Particle Separation

100 g of nanoporous silica particles (preparable and/or prepared as described in Examples 1 and/or 2 above) were fed into an air classifier (TTS, Hosokawa-Alpine), with the air flow adjusted from 53 to 42 m³/h and the speed set at between 2,475 and 13,500 rpm. 11 g of fines and 8 g of coarse materials were collected. The particle size distribution calculated as (D₉₀/D₁₀) was reduced from 4.5 to 1.8.

### Example 4

### Nanoporous Silica Particle Properties

Two porous silica particle types with different porosities and densities (1.2 mL/g and 0.9 mL/g, and 0.18 mg/mL and 0.33 mg/mL, respectively) were characterised to determine their MMAD and GSD using an 8-Stage Cascade Impactor (Marple), as shown in Table 1 below.

TABLE 1

| | Bulk | | | | | | |
|---|---|---|---|---|---|---|---|
| | Density | D₁₀ | D₅₀ | D₉₀ | MMAD | | GMPS |
| | (mg/mL) | (μm) | (μm) | (μm) | (μm) | PSD | (μm) |
| Silica 1 | 0.18 | 3.0 | 4.1 | 5.5 | 4.33 | 1.97 | 3.8 |
| Silica 2 | 0.33 | 2.6 | 3.8 | 5.1 | 3.6 | 1.8 | 4.1 |

By optical observation, it could be seen that, in small quantities, the above particles aggregated and had poor flow properties.

Attempts were made to reduce particle aggregation for formulation by using the well-known glidant magnesium stearate. This is an approved excipient for inhalation products. The particles were mechanically mixed with magnesium stearate (Sigma) at several different weight ratios in the range 1-5% magnesium stearate, but the flow properties of the particles did not improve.

### Example 5

### Loading of Thalidomide in Silica

Thalidomide was encapsulated into the porous silica particles of Example 4 above (those with the bulk density of 0.18 mg/mL and the MMAD of 4.33 μm) by a solvent impregnation and evaporation method. A concentrated solution of thalidomide was made in a chosen good solvent for the drug, and various known masses of nanoporous silica particles were added to the solution. The solvent was removed by evaporation.

In one example, thalidomide (200 mg; Sigma) was dissolved in methylene chloride (120 mL; Sigma) at room temperature in a round-bottomed flask. Nanoporous silica particles (300 mg) were added to the thalidomide solution.

The mixture was stirred for 30 minutes at 40° C. The solvent was evaporated with controlled evaporation under a reduced pressure of 200 mBar in a rotary evaporator, with a water bath temperature of 60° C. The resultant dry powder that was collected was free flowing. The samples were further dried at 40° C. under vacuum for 4 hours.

The samples were characterized by TGA to evaluate drug loading. Loading amounts of 20, 40 and 50 wt % (calculated as mass of drug/mass of loaded particles) were determined as shown in FIG. 1.

The physical state of the drug (crystalline vs amorphous) was measured with DSC and is shown in FIG. 2. Thalidomide has a melting peak in DSC of around 270° C. It can be seen from FIG. 2 that thalidomide was stabilized in an amorphous state in the samples with a drug loading up to 40%. There was a small melting peak observed with the samples with a drug loading amount of 50% indicating presence of crystalline drug. This demonstrates that the maximum loading capacity of the particles was probably reached at 50% loading i.e. complete loading of the particles and it is likely that crystalline drug resided on the outside of the particles.

Analysis by light microscopy showed that the free drug is fully encapsulated in the porous silica particles. This is shown in FIG. 3 (free thalidomide on the left, and thalidomide-loaded silica particles (20% loading) on the right).

### Example 6

### Dissolution Kinetics of Thalidomide-Loaded Silica Particles I

The dissolution kinetics of the 3 different loading samples described in Example 5 were characterized in SLF (pH 7.4; made up with the salts NaCl, NaHCO₃, KCl, MgCl₂, CaCl₂, Na₂SO₄, sodium citrate dihydrate, NaH₂PO₄ (all from Sigma)) at 37° C. using a USP2 dissolution apparatus with stirring speed 50 rpm. Free, unloaded thalidomide was used as control. Concentration of drug at set times after release was measured by a UV/vis spectrometer (Cecil 3021) at 220 nm.

The dose of thalidomide was calculated as 5 mg of thalidomide in 500 mL of SLF. The data are shown in FIG. 4. The dissolution kinetics of thalidomide was dramatically enhanced for all of silica-loaded samples compared to free drug control.

The effective solubility of thalidomide was also dramatically enhanced due to the stabilization of a supersaturated concentration of the drug. Cₘₐₓ was achieved in 10 minutes with all formulations in which thalidomide was loaded into silica particles compared to 30 minutes for the free drug. The AUC was doubled with all of the silica formulations.

### Example 7

### Dissolution Kinetics of Thalidomide-Loaded Silica Particles II

In order to check API chemical stability, release kinetics in PBS (pH 7.4; Sigma) were tested at 37° C. with the stirring speed set at 50 rpm using a USP2 apparatus.

Drug concentration after release was measured by HPLC (Agilent 1100).

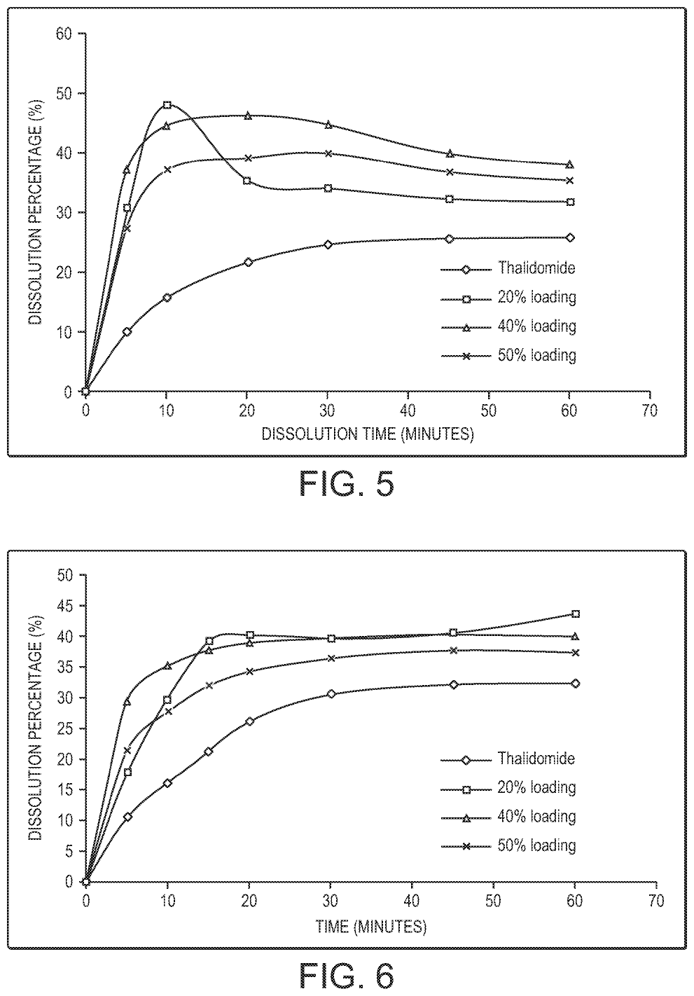

Again, the dose was of thalidomide was calculated as 5 mg of thalidomide in 500 mL of PBS. The data are shown in FIG. 5. The dissolution kinetics of thalidomide showed similar profiles to the dissolution in SLF with all of the thalidomide-loaded silica particles having faster dissolution kinetics and higher effective solubility compared with free thalidomide.

### Example 8

### Dissolution Kinetics of Thalidomide-Loaded Silica Particles III

Given that thalidomide rapidly recrystallizes in solution, an experiment was conducted in the presence of a surfactant to stabilize the dissolved drug.

Tween 80 (1 wt %; Sigma) was added in the dissolution bath in SLF media. The dosage was increased to 80 mg to investigate the dissolution kinetics at high drug concentration, to simulate dissolution in non-sink conditions.

The dissolution kinetics is shown in FIG. 6. The dissolution of the silica loaded particles was significantly enhanced reaching a Cₘₐₓ in around 10 to 15 minutes compared to 30 minutes for the free drug. The dissolved amount of thalidomide was doubled for the silica loaded particles.

### Example 9

Local Irritation of Inhalation of Silica Loaded with 40% (Weight) Thalidomide in Man

Two Sincapsules® containing 20 mg of sodium cromoglycate (Sanofi AB) were opened and emptied using pressurized air. One of the capsules was filled with 2.5 mg of silica particles loaded with 40% (wt %) of thalidomide, obtained as described in Example 5 above and the other was loaded with the same amount of unloaded silica particles as a control.

The Sincapsules were inspected before use. It was clear that unloaded silica particles were sticking to the capsule wall (FIG. 7, left-hand capsule). Surprisingly, silica particles loaded with 40% (weight) thalidomide were nonadherent and moved around freely when moving the capsule (FIG. 7, right-hand capsule). Thus, the loading of thalidomide into the silica particles improved the flow properties of the particles without the addition of further excipients.

The closed capsules were loaded in a Intal® Spinhaler® according to the manufacturer's instructions in the packaging insert. The full doses were inhaled by one healthy volunteer, following the instructions in the packaging insert, making sure that the full content was inhaled.

Local irritation in the upper airways and taste was assessed on a 10-graded scale (0-10, where 0 is no irritation/taste and 10 the worst irritation/taste one could think of) immediately after inhalation and at 5 and 60 minutes post inhalation.

The local irritation in the upper airways (both capsules) was 0 at time point 0, 5 and 60 min after inhalation. The taste sensation (both capsules) was 0 at time point 0, 5 and 60 min after inhalation.

It was concluded that inhaled silica loaded with 40% (weight) thalidomide does not cause local irritation or taste sensation in man.

1. A pharmaceutical composition suitable for administration to the lung, which composition comprises a plurality of amorphous nanoporous silica particles, in which one or more immunomodulatory imide drug is loaded into the pores of said particles, and wherein the silica particles have:
(a) a mass median aerodynamic diameter that is between about 0.1 μm and about 10 μm; and
(b) a geometric standard deviation that is less than about 4, wherein the immunomodulatory imide drug is effective in the treatment of an interstitial lung disease following pulmonary administration.

2. The composition as claimed in claim 1, wherein the loaded silica particles have a mass density that is less than about 0.4 g/cm³.

3. The composition as claimed in claim 1, wherein the mass median aerodynamic diameter is between about 3 μm and about 5 μm.

4. The composition as claimed in claim 1, wherein the geometric standard deviation is between about 1 and about 1.5.

5. The composition as claimed in claim 1, wherein the silica particles have a pore size that is between about 10 nm and about 20 nm.

6. The composition as claimed in claim 1, wherein the silica particles have a pore volume that is between 0.2 and 3 cm³/g.

7. The composition as claimed in claim 1, wherein the silica particles have a surface area that is between about 150 and about 1200 m²/g.

8. The composition as claimed in claim 1, wherein the silica particles are essentially spherical.

9. The composition as claimed in claim 1, wherein up to about 45% of the total weight of the loaded particles is immunomodulatory imide drug.

10. The composition as claimed in claim 1, wherein the immunomodulatory imide drug is essentially amorphous.

11. The composition as claimed in claim 1, wherein the silica particles consist essentially of a synthetic biodegradable amorphous mesoporous silica.

12. The composition as claimed in claim 1, wherein the immunomodulatory imide drug is thalidomide.

13. A process for the production of a composition, which process comprises:
(a) separating silica particles to obtain particles having a mass median aerodynamic diameter that is between about 0.1 μm and about 10 μm and a geometric standard deviation that is less than about 4; followed by
(b) loading the obtained particles with an immunomodulatory imide drug to form the composition according to claim 1.

14. The process as claimed in claim 13, wherein the silica particles are separated and classified into the desired particle size range using an air classifier.

15. The process as claimed in claim 13, wherein the silica particles are loaded with one or more immunomodulatory imide drug using a process of solvent evaporation.

16. The process as claimed in claim 13, wherein the silica particles are manufactured by reacting tetraethyl orthosilicate with a template made of micellar structures.

17. The process as claimed in claim 13, wherein the silica particles are manufactured by a sol-gel method comprising a condensation reaction of an aqueous suspension of silica nanoparticles with a non-miscible organic solution, oil, or liquid polymer, followed by gelation by means of change in pH and/or evaporation of the aqueous phase.

18. A pharmaceutical composition prepared according to the process of claim 13.

19. The pharmaceutical formulation comprising a composition as defined in claim 1 in admixture with one or more pharmaceutically-acceptable excipients.

20. The pharmaceutical formulation as claimed in claim 19, wherein the excipient is a hydrocarbon, a fluorocarbon and/or a hydrogen-containing fluorocarbon propellant.

21. A process for the production of a pharmaceutical formulation as defined in claim 19, which process comprising admixing the composition with the one or more pharmaceutically-acceptable excipients to form the pharmaceutical composition.

22. (canceled)

23. A method of treatment of an interstitial lung disease, which method comprises the pulmonary administration of a pharmacologically-effective amount of a composition as defined in claim 1, to a patient in need of such treatment.

24. The method of treatment as defined in claim 23, wherein the interstitial lung disease is idiopathic pulmonary fibrosis.

25. The method of treatment as defined in claim 23, wherein the interstitial lung disease is sarcoidosis.
